(19) United States
(12) Patent Application Publication
(10) Pub. No.: US 2017/0316156 A1
(43) Pub. Date: Nov. 2, 2017

(54) INFORMATION PROCESSING APPARATUS, INFORMATION PROCESSING SYSTEM, INFORMATION PROCESSING METHOD, AND RECORDING MEDIUM

(71) Applicant: CANON KABUSHIKI KAISHA, Tokyo (JP)

(72) Inventor: Keisuke Arima, Yokohama-shi (JP)

(21) Appl. No.: 15/494,344

(22) Filed: Apr. 21, 2017

(30) Foreign Application Priority Data
Apr. 28, 2016 (JP) 2016-091610

(51) Int. Cl. G06F 19/00; A61B 6/00

(52) U.S. Cl. CPC G06F 19/321; A61B 6/52

(57) ABSTRACT

An information processing apparatus acquires a request for re-imaging or a request for additional imaging. The request for re-imaging is information for requesting imaging for acquiring a second medical image as re-imaging for a previously acquired first medical image. The request for additional imaging is information for requesting imaging for additionally acquiring a third medical image. The information processing apparatus attaches, to the second medical image, information for identifying the first medical image as information for identifying the second medical image when the request for re-imaging is acquired and attaches, to the third medical image, information for identifying the third medical image when the request for additional imaging is acquired.

### BACKGROUND

### Field

The present disclosure relates to an information processing apparatus, an information processing system, an information processing method, and a recording medium.

### Description of the Related Art

Various medical images are utilized for medical diagnosis and treatment, and information systems that enable management and utilization of such medical images have been built. Users sometimes make a request for re-imaging or additional imaging in relation to, for example, an acquired medical image by using such information systems because imaging has been unsuccessful. Japanese Patent Laid-Open No. 2011-212224 discloses the following configuration. An image processing apparatus transmits a re-imaging request regarding an image for which re-imaging is needed to a radiology information system (RIS), which is a system for managing imaging orders. The RIS then adds a re-imaging order.

If an instruction for re-imaging or additional imaging is made via the RIS, there may be confusion about whether the requested imaging is performed in a new examination to be carried out or in a previously started examination as re-imaging or additional imaging. Consequently, appropriate supplementary information can fail to be attached to an acquired medical image.

### SUMMARY OF THE INVENTION

According to some embodiments, an information processing apparatus includes a first acquiring unit and an attaching unit. The first acquiring unit is configured to acquire a request for re-imaging or a request for additional imaging. The request for re-imaging is information for requesting imaging for acquiring a second medical image as re-imaging for a previously acquired first medical image, whereas the request for additional imaging is information for requesting imaging for additionally acquiring a third medical image. The attaching unit is configured to attach, to the second medical image, information for identifying the first medical image for which the request for re-imaging has been made as information for identifying the second medical image in a case where the request for re-imaging is acquired by the first acquiring unit, and to attach, to the third medical image, information for identifying the third medical image in a case where the request for additional imaging is acquired by the first acquiring unit.

Further features will become apparent from the following description of exemplary embodiments with reference to the attached drawings.

### BRIEF DESCRIPTION OF THE DRAWINGS

FIG. 1 is a diagram illustrating an example of an information system including an information processing apparatus according to a first embodiment.

FIG. 2 is a diagram illustrating an example of a hardware configuration of the information processing apparatus according to the first embodiment.

FIG. 3 is a diagram illustrating an example of a functional configuration of the information processing apparatus according to the first embodiment.

FIG. 4 is a diagram illustrating an example of a graphical user interface (GUI) provided by the information processing apparatus according to the first embodiment.

FIG. 5 is a diagram illustrating an example of a GUI provided by an external apparatus connected to the information processing apparatus according to the first embodiment.

FIG. 6 is a flowchart illustrating an example of a process performed by the information processing apparatus according to the first embodiment.

FIG. 7 is a flowchart illustrating an example of a process performed by the information processing apparatus according to the first embodiment.

FIG. 8 is a flowchart illustrating an example of a process performed by an information processing apparatus according to a second embodiment.

FIG. 9 is a flowchart illustrating an example of a process performed by an information processing apparatus according to a third embodiment.

### DESCRIPTION OF THE EMBODIMENTS

Embodiments will be described below with reference to the accompanying drawings.

### First Embodiment

Medical images are utilized by doctors to check the condition of patients or to make a diagnosis. Medical imaging apparatuses (hereinafter, referred to as modalities) are used to acquire medical images. Examples of modalities include apparatuses such as a magnetic resonance imaging (MRI) apparatus, an X-ray computed tomography (CT) apparatus, an ultrasound imaging apparatus, a photoacoustic tomography (PAT) apparatus, a positron emission tomography (PET)/single photon emission computed tomography (SPECT) apparatus, an optic coherence tomography (OCT) apparatus, and an X-ray imaging apparatus (e.g., a digital radiography (DR) apparatus). Medical images acquired by such modalities include various kinds of images ranging from those describing morphologic features to those describing functional values, for example, the blood flow rate in the brain. Doctors select an appropriate modality in accordance with the condition of each patient to acquire medical images.

To carry out examinations using these modalities, information is communicated via an intra-hospital information system and a network. The intra-hospital information system includes a hospital information system (HIS) that manages information, such as patient information and examination request information. The patient information includes the name and the ID of each patient, for example. The examination request information includes the date and time of each examination and details of imaging, for example. The intra-hospital information system also includes a radiology information system (RIS) that manages information, such as patient information and examination request information relating particularly to the radiology department.

In addition, a picture archiving and communication system (PACS) is a system for storing and displaying acquired medical images enabling use of such medical images for making a diagnosis. Acquired medical images are sometimes transferred to the PACS after medical technicians have confirmed, using a quality assurance apparatus, that the acquired medical images have sufficiently high quality for interpretation by doctors, instead of being transferred to the PACS without any check. Digital Imaging and Communications in Medicine (DICOM) is used as a standard for information communication between apparatuses and modalities via a network in such intra-hospital information systems.

Various kinds of information are exchanged in intra-hospital information systems. If it takes some time to contact an imaging technician when re-imaging or additional imaging is desired, the quality assurance technician or doctor may feel it is troublesome to make such contact. Thus, an instruction for re-imaging or additional imaging can be made quickly via apparatuses in intra-hospital information systems. In addition, the consistency of information used in intra-hospital information systems is maintained at the time of re-imaging or additional imaging. An information processing apparatus according to a first embodiment handles such issues.

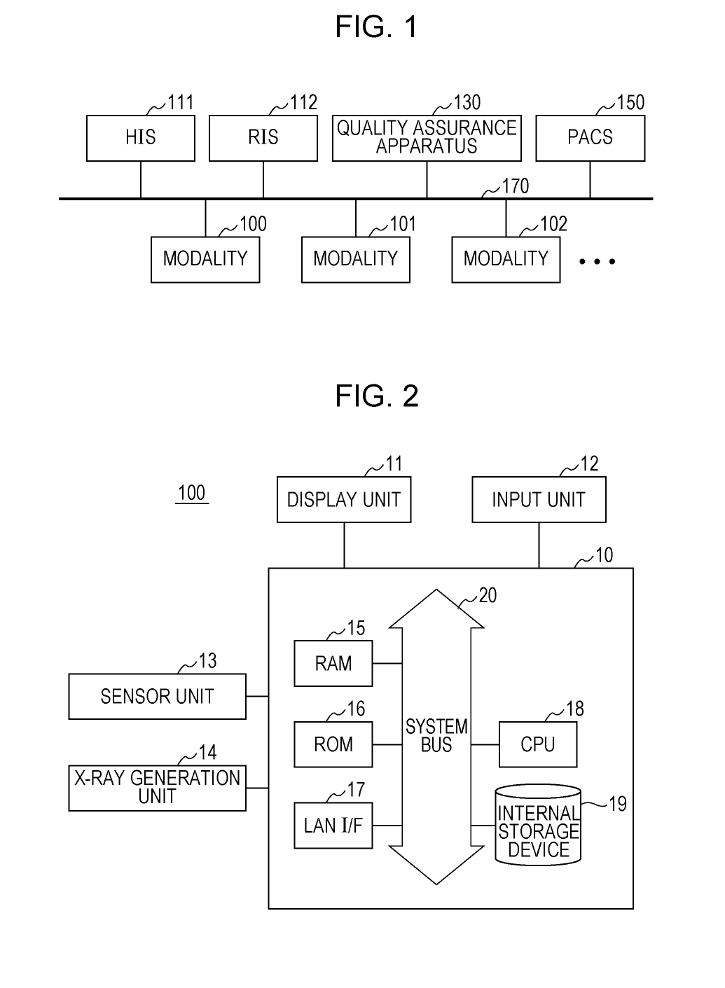

FIG. 1 is a diagram illustrating an example of an intra-hospital information system including modalities such as modalities 100, 101, and 102 and apparatuses that are connected to the modalities 100, 101, and 102 via a network 170 to be able to perform communication. As illustrated in FIG. 1, the modalities 100, 101, and 102 are connected to an HIS 111, an RIS 112, a quality assurance apparatus 130, and a PACS 150 via the network 170. The intra-hospital information system can include a plurality of modalities as illustrated in FIG. 1 or can include a single modality. The modalities 100, 101, and 102 can be modalities of a single kind or modalities of different kinds. The network 170 is based on, for example, Ethernet.

Each of the modalities 100, 101, and 102 can be, for example, one of modalities such as an MRI apparatus, an X-ray CT apparatus, an ultrasound imaging apparatus, a PAT apparatus, a PET/SPECT apparatus, an OCT apparatus, and a DR apparatus. In the first embodiment, a description of an example case is provided where the modality 100 is a DR apparatus. An information processing apparatus according to the first embodiment is an imaging control apparatus 10 that controls imaging performed by the modality 100. The imaging control apparatus 10 of the modality 100 will be described in detail below.

Based on the search conditions set by an imaging technician, the modalities 100, 101, and 102 acquire examination request information that meets the search conditions from the RIS 112. The modalities 100, 101, and 102 analyze the acquired examination request information. The imaging technician then selects an examination to be carried out. The modalities 100, 101, and 102 notify the RIS 112 of the start of the examination for which an examination start instruction has been provided by the imaging technician. Then, the modalities 100, 101, and 102 radiate X-rays in accordance with an imaging instruction provided by the imaging technician and acquire a medical image.

This processing is repeatedly performed in accordance with the details of the examination. After all the imaging operations included in the examination have been completed, the imaging technician provides an examination end instruction. The modalities 100, 101, and 102 create an examination completion notification and output the examination completion notification to the RIS 112. The modalities 100, 101, and 102 also output the acquired medical image(s) to the quality assurance apparatus 130. The quality assurance technician checks each check-target image by using the quality assurance apparatus 130 and determines whether the image has sufficiently high quality for interpretation by doctors.

The HIS 111 registers patient information and creates examination request information. The HIS 111 transmits the created examination request information to the RIS 112. The HIS 111 receives an examination start notification and an examination completion notification from the modalities 100, 101, and 102 via the RIS 112. Upon receipt of an examination completion notification, the HIS 111 performs control so as to allow an intra-hospital accounting system to perform an accounting process by using examination end information attached to the examination completion notification.

The RIS 112 attaches information necessary for an examination, such as information regarding a site to be examined, to examination request information received from the HIS 111 and transmits the resultant examination request information to one of the modalities 100, 101, and 102 corresponding to the examination request information. The RIS 112 also receives an examination start notification and an examination completion notification from, for example, the modality 100 and transmits the received examination start notification and the examination completion notification to the HIS 111. The RIS 112 can understand the status of each examination which the modality 100 or the like has been requested to perform. The RIS 112 is an example of a management apparatus that manages information regarding an examination.

The quality assurance apparatus 130 includes an image server (not illustrated) that stores image data transmitted from the modalities 100, 101, and 102. The quality assurance apparatus 130 also includes a monitor (not illustrated) that acquires image data from the image server and displays an image based on the image data and a control device (not illustrated) on which an application of an image viewer used to perform image processing and display an image on the monitor runs. The quality assurance apparatus 130 can store received image data in the control device or on an external recording medium of the quality assurance apparatus 130. The quality assurance apparatus 130 also functions as a medical image viewer that receives image data transmitted from the modalities 100, 101, and 102 and displays an image based on the image data.

The quality assurance technician checks, by using the quality assurance apparatus 130, image data acquired through an examination. The quality assurance technician checks, for example, whether the density and the contrast of a medical image, the position and content of an annotation of the medical image, content of a DICOM tag, and image processing are appropriate. The quality assurance technician corrects the image data if necessary. The quality assurance technician also determines whether positioning of the patient is appropriate in the image data, whether the dose is appropriate, whether the imaging conditions are appropriate, and whether another imaging is needed to identify the lesion.

If the positioning of the patient or the dose is inappropriate, the quality assurance technician requests the modality 100 to perform re-imaging. If the quality assurance technician determines that another imaging is needed, the quality assurance technician requests the modality 100 to perform additional imaging. After completion of the re-imaging or additional imaging requested by the quality assurance technician based on the operation for quality assurance, the modality 100 transmits acquired image data to the quality assurance apparatus 130. The quality assurance technician checks, by using the quality assurance apparatus 130, the image data acquired through the re-imaging or additional imaging. If the quality assurance technician determines that the image data is appropriate, the quality assurance technician performs an operation to complete the examination. The quality assurance technician then transfers the image data to the PACS 150.

The PACS 150 stores image data received from the quality assurance apparatus 130. The PACS 150 includes a monitor (not illustrated) that displays an image based on image data, and a control device (not illustrated) that manages the PACS 150. Doctors interpret each image based on corresponding image data by using the PACS 150 or an apparatus connected to the PACS 150 via the network 170. To interpret an image based on image data is to acquire information used for medical diagnosis and treatment of a corresponding patient by observing the image based on the image data. Since the quality assurance technician has adjusted the position and content of an annotation of the image data, the content of the DICOM tag, the positioning of the target site of the patient, and the image processing appropriately in advance, the doctors can efficiently interpret the image.

A description will now be provided of patient information, examination request information, executed examination information, and examination end information that are exchanged between the apparatuses of the intra-hospital information system according to the first embodiment illustrated in FIG. 1. These four kinds of information are sometimes collectively referred to as examination information in the first embodiment.

The patient information is information regarding a patient who is a subject of an examination performed by the modality 100, for example. The patient information includes information, such as the patient ID that is a unique ID for uniquely identifying a patient (subject) and the name, the sex, the date of birth, and the age of the patient. The patient information is typically input and stored by using the HIS 111. However, the patient information can be input, corrected, and stored by using the RIS 112 and the modality 100. The patient information can be stored in an information storage device, such as a server, connected to the network 170. Such a configuration enables the patient information input by using the individual apparatuses to be shared with the consistency thereof being assured.

The examination request information is specific information used to perform an examination, such as the date and time of the examination, the modality used to perform the examination, the technician who carries out the examination, and the imaging procedure used for imaging included in the examination. Specifically, the examination request information includes information, such as the examination ID that is a unique ID for uniquely identifying the examination, the scheduled date and time of the examination, the apparatus scheduled to be used for the examination, and the specific imaging procedure scheduled to be performed in the examination. The examination request information also includes the name of the imaging technician who carries out the examination, the names of the department and the doctor having issued the request for the examination, and the patient information described above.

In the case where the HIS 111, the RIS 112, and the modality 100 conform to the DICOM standard, the examination request information includes tag information that is defined by the MODALITY WORK LIST SOP class of the DICOM standard. The examination request information is typically input and stored by using the HIS 111. However, the examination request information can be input, corrected, and stored by using the RIS 112 and the modality 100. In this case, the HIS 111 is notified of the examination request information at a given timing. The examination request information can be stored in an information storage device, such as a server, connected via the network 170. This configuration enables the examination request information to be shared among the apparatuses.

The executed examination information is specific information regarding imaging performed during an examination that has been finished. The executed examination information includes information, such as the imaging procedure that has been performed, the number of acquired images, duration of the examination, the presence or absence of imaging additionally performed during the examination, the name of the imaging technician who has carried out the examination, and the radiation dose of the patient during the examination. The information regarding the imaging procedure that has been performed includes the name of the imaging procedure, a site (including distinction of the left and right sides) that has been imaged by using the imaging procedure, the imaging direction, the orientation of the body of the patient, and the imaging method used for the imaging procedure. Examples of the imaging method used for the imaging procedure include still-image-based imaging, moving-image-based imaging, and angiography-based imaging. The executed examination information is created by the modality that has performed the examination.

The examination end information is information indicating content that has been actually performed in response to examination request information selected by the imaging technician by using the modality 100. The examination end information includes information, such as the examination ID that is a unique ID for uniquely identifying the examination, the execution date and time of the examination, the modality that has performed the examination, and the name of the imaging technician who has carried out the examination. The examination end information includes the patient information and the executed examination information described above. In the case where the HIS 111, the RIS 112, and the modality 100 conform to the DICOM standard, the examination end information includes tag information defined by the MODALITY PERFORMED PROCEDURE STEP SOP class of the DICOM standard.

The examination end information is created when the imaging technician has finished all the requested examination operations by using the modality 100 or when an instruction to transmit examination end information is given after the examination has been finished. Then, the modality 100 transmits an examination completion notification including the examination end information to the RIS 112. The RIS 112 then transmits the examination end information to the HIS 111, and the examination end information is used in a workflow, such as the accounting process, performed after the end of the examination.

FIG. 2 is a diagram illustrating an example of a hardware configuration of the information processing apparatus according to the first embodiment. The information processing apparatus according to the first embodiment is the imaging control apparatus 10 of the modality 100. The modality 100 includes the imaging control apparatus 10, a display unit 11, an input unit 12, a sensor unit 13, and an X-ray generation unit 14.

The imaging control apparatus 10 includes a random access memory (RAM) 15, a read-only memory (ROM) 16, a local area network interface (LAN I/F) 17, a central processing unit (CPU) 18, and an internal storage device 19, which are connected to one another via a system bus 20. The imaging control apparatus 10 is, for example, a computer. Each of the RAM 15, the ROM 16, and the internal storage device 19 is an example of a memory, and the CPU 18 is an example of a processor. The imaging control apparatus 10 can include a plurality of CPUs.

The display unit 11 and the input unit 12 are connected to the imaging control apparatus 10. The display unit 11 is, for example, a liquid crystal display. The display unit 11 displays an image based on image data and a GUI. The input unit 12 is, for example, an input device such as a mouse, a keyboard, or a barcode reader. The imaging technician can input various commands or data to the imaging control apparatus 10 by using the input unit 12. The display unit 11 and the input unit 12 can be integrated into a touch panel monitor, for example.

The sensor unit 13 generates image data for a patient who is a subject irradiated with X-rays from the X-ray generation unit 14 under the control of the imaging control apparatus 10. The image data is transmitted to the imaging control apparatus 10. The X-ray generation unit 14 irradiates the subject with X-rays under the control of the imaging control apparatus 10.

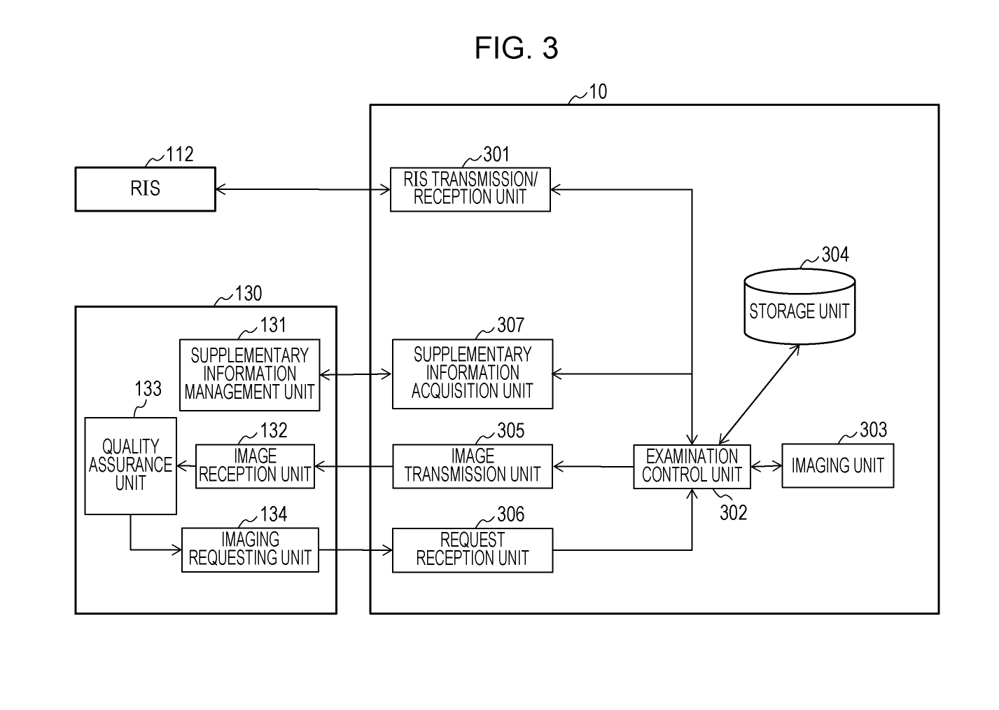

FIG. 3 is a diagram illustrating an example of a functional configuration of the imaging control apparatus 10. FIG. 3 illustrates the configurations of the RIS 112 and the quality assurance apparatus 130 as well as the configuration of the imaging control apparatus 10 in order to describe functions of the imaging control apparatus 10 according to the first embodiment. Functions (described below) of the imaging control apparatus 10 are implemented as a result of the CPU 18 executing a program stored in the ROM 16 or the internal storage device 19.

An RIS transmission/reception unit 301 communicates with the RIS 112. Specifically, the RIS transmission/reception unit 301 receives examination request information from the RIS 112. The RIS transmission/reception unit 301 also transmits examination end information to the RIS 112.

An examination control unit 302 causes the display unit 11 to display a GUI in accordance with the progress of the examination. The examination control unit 302 acquires an input operation performed for the GUI from the input unit 12 and performs control so that various instructed functions can be performed. The examination control unit 302 also performs image processing on acquired image data and attaches supplementary information to the image data based on the examination request information. That is, the examination control unit 302 is an example of an attaching unit. The supplementary information is, for example, the image ID for uniquely identifying the medical image, the examination ID for uniquely identifying the examination, and various kinds of information defined by the DICOM standard.

The examination control unit 302 stores the image data in a storage unit 304 together with the supplementary information. The examination control unit 302 also determines whether the current request is a request for re-imaging or a request for additional imaging based on the examination request information received by a request reception unit 306 (described below). That is, the examination control unit 302 is an example of a determining unit. The examination control unit 302 also generates examination information used to perform re-imaging or additional imaging. That is, the examination control unit 302 is an example of a generating unit. The examination control unit 302 can implement the function of the generating unit, the function of the attaching unit, and the function of the determining unit by using a plurality of modules.

An imaging unit 303 performs imaging corresponding to examination request information received from the RIS 112 under the control of the examination control unit 302. The imaging unit 303 controls the sensor unit 13 and the X-ray generation unit 14 and performs imaging. The imaging unit 303 acquires image data from the sensor unit 13. That is, the imaging unit 303 is an example of an image acquiring unit.

The storage unit 304 stores information regarding examinations performed by the modality 100, acquired image data, and supplementary information. An image transmission unit 305 transmits the image data acquired by the modality 100 to an external apparatus via the network 170. The external apparatus can be, for example, the quality assurance apparatus 130 or the PACS 150 in the first embodiment. That is, the image transmission unit 305 is an example of an output unit.

The request reception unit 306 receives an examination request for re-imaging or additional imaging from an external apparatus. The external apparatus is, for example, the quality assurance apparatus 130 or the PACS 150. That is, the request reception unit 306 is an example of a first acquiring unit.

A supplementary information acquisition unit 307 acquires supplementary information from an external apparatus. For example, the supplementary information acquisition unit 307 requests the quality assurance apparatus 130 to transmit supplementary information regarding certain image data or a certain examination. Then, the supplementary information acquisition unit 307 receives supplementary information transmitted from the external apparatus in response to the request. The supplementary information is, for example, the image ID for uniquely identifying the image data or the examination ID for uniquely identifying the examination. The external apparatus requested to transmit the supplementary information by the supplementary information acquisition unit 307 is, for example, the quality assurance apparatus 130, the PACS 150, or the RIS 112. When transmitting the request, the supplementary information acquisition unit 307 also transmits information to be used by the external apparatus to search for the supplementary information to be acquired.

The functions of the imaging control apparatus 10 illustrated in FIG. 3 will be described in detail in accordance with the flow of acquiring an X-ray image.

A plurality of pieces of examination request information received by the RIS transmission/reception unit 301 from the RIS 112 are sent to the storage unit 304 and are stored in the storage unit 304 by the examination control unit 302. The examination control unit 302 causes the display unit 11 to display the plurality of pieces of examination request information received from the RIS 112. In accordance with a method for displaying a plurality of pieces of examination request information, a list of examinations that have not been completed is displayed from among examinations indicated by the pieces of examination request information received from the RIS 112, for example.

The imaging technician selects, by using the input unit 12, a piece of examination request information corresponding to an examination to be performed and starts the examination. As described above, the examination request information includes information regarding a specific imaging procedure to be performed in the examination. The imaging procedure is set by selecting an imaging protocol. The imaging protocol is stored in the storage unit 304 as default values of imaging conditions, image processing parameters, or the like for each imaging procedure. The imaging technician can perform imaging after changing the default values by using the input unit 12. The information input by the imaging technician is sent to the imaging unit 303 via the examination control unit 302.

The imaging technician selects a piece of examination request information from the list displayed on the display unit 11 and presses an examination start button, which is a GUI displayed on the display unit 11. Then, the first imaging protocol is selected from among imaging protocols included in the piece of examination request information. The imaging unit 303 controls the X-ray generation unit 14 so that the X-ray generation unit 14 is ready to radiate X-rays in imaging conditions determined by the first imaging protocol. The imaging conditions of the X-ray generation unit 14 include information regarding an X-ray tube current, an X-ray tube voltage, an irradiation period, and a distance between the patient and the X-ray tube.

In addition, the imaging technician adjusts the position/orientation of the subject and adjusts the diaphragm of the X-ray tube of the X-ray generation unit 14. If attachment of a grid is required by the imaging procedure to be performed, the imaging technician checks whether the grid is attached to the sensor unit 13. After all the preparations have been done, the imaging technician presses an X-ray irradiation switch (not illustrated) of the X-ray generation unit 14 to irradiate the subject with X-rays and acquires X-ray image data for the subject.

After acquisition of the image data has completed, the imaging unit 303 notifies the examination control unit 302 that imaging based on the first imaging protocol has completed. The examination control unit 302 corrects the acquired image data and performs image processing by using image processing parameters set by the imaging protocol. The examination control unit 302 then causes the display unit 11 to display an image based on the image data and stores the image data in the storage unit 304. The examination control unit 302 attaches, to the image data, the image ID that is a unique ID for uniquely identifying the image or the protocol ID for uniquely identifying the imaging protocol corresponding to the imaging procedure that has been performed.

After the imaging based on the first imaging protocol has completed, the state transits to a state where the second imaging protocol is selected by the examination control unit 302. Imaging is also performed for the second imaging protocol just like the imaging based on the first one. In this way, the imaging technician performs imaging based on all the requested imaging protocols. After imaging based on all the imaging protocols have completed, the imaging technician presses an examination end button 413, which is a GUI displayed on the display unit 11, and ends the examination.

The examination control unit 302 detects an input operation performed on the examination end button 413 and sends the acquired image data to the image transmission unit 305. The image transmission unit 305 then transmits the image data to an external apparatus. In the first embodiment, the image transmission unit 305 transmits the image data to the quality assurance apparatus 130, which is an example of an external apparatus, and the image data is used by the quality assurance technician for quality assurance. The image transmission unit 305 can cause the display unit 11 to display a GUI that enables the imaging technician to select an apparatus to which the image data is to be transmitted and can transmit the image data in accordance with an input operation performed on the GUI.

When the examination is finished, the examination control unit 302 creates examination end information and causes the RIS transmission/reception unit 301 to transmit an examination completion notification including the examination end information to the RIS 112.

The quality assurance apparatus 130 includes, for example, a supplementary information management unit 131, an image reception unit 132, a quality assurance unit 133, and an imaging requesting unit 134 as the functional units thereof.

The supplementary information management unit 131 searches for supplementary information requested by the imaging control apparatus 10 of the modality 100, for example. If the quality assurance apparatus 130 stores the requested supplementary information, the supplementary information management unit 131 transmits the supplementary information to the imaging control apparatus 10.

The image reception unit 132 receives image data from an external apparatus. The external apparatus from which the quality assurance apparatus 130 receives image data is, for example, the modality 100 or the PACS 150. An image based on the image data received by the image reception unit 132 is displayed on the monitor of the quality assurance apparatus 130 and is used by the quality assurance technician for quality assurance.

The quality assurance unit 133 finalizes the result of the quality assurance operation in accordance with an input operation performed by the quality assurance technician. For example, in response to the quality assurance technician pressing, for example, an image finalize button 511, which is a GUI displayed on the monitor of the quality assurance apparatus 130, the quality assurance unit 133 finalizes the result of the quality assurance operation performed on the image data. The quality assurance technician makes a correction by using the quality assurance apparatus 130 if necessary and finalizes the result of the quality assurance operation performed on the image data when determining that the image data represents an image suitable for medical diagnosis and treatment.

The imaging requesting unit 134 requests the modality 100 to perform re-imaging or additional imaging in relation to an image that has been subjected to the quality assurance operation. The imaging requesting unit 134 transmits information indicating a request for re-imaging or additional imaging to the modality 100 in response to an input operation performed on a re-imaging request button 512 or an additional imaging request button 513, which is a GUI displayed on the monitor of the quality assurance apparatus 130. Information indicating a request for re-imaging is hereinafter referred to as a re-imaging request. In addition, information indicating a request for additional imaging is hereinafter referred to as an additional imaging request. The modality that is requested to perform re-imaging or additional imaging is a modality that has acquired the image that has been subjected to the quality assurance operation or another modality capable of performing the requested imaging.

The functions of the imaging control apparatus 10 and the quality assurance apparatus 130 illustrated in FIG. 3 will now be described in detail in accordance with a flow of performing the quality assurance operation on an X-ray image and making a request for re-imaging or additional imaging.

If the examination end button 413, which is a GUI displayed on the display unit 11 of the modality 100, is pressed by the imaging technician, the image transmission unit 305 transmits the image data to the quality assurance apparatus 130. The image reception unit 132 of the quality assurance apparatus 130 receives the image data and stores the image data in a storage medium (not illustrated) of the quality assurance apparatus 130. The quality assurance unit 133 causes an image based on the image data received by the image reception unit 132 to be displayed on a monitor of the quality assurance apparatus 130 so as to allow the quality assurance technician to perform the quality assurance operation on the image.

The quality assurance technician sometimes determines that re-imaging or additional imaging is needed because positioning of the patient or the X-ray dose is inappropriate. In such a case, the quality assurance technician presses the re-imaging request button 512 or the additional imaging request button 513, which is a GUI displayed on the monitor of the quality assurance apparatus 130. In response to an input operation performed on such a GUI, the imaging requesting unit 134 transmits a request for re-imaging or additional imaging to the modality 100 that has acquired the image data that has been subjected to the quality assurance operation. The request for re-imaging includes the examination ID and the image ID for identifying the acquired image for which re-imaging is to be performed. In addition, the request for additional imaging includes a protocol ID for identifying the imaging protocol that identifies the imaging procedure used for additional imaging.

The request reception unit 306 of the modality 100 receives the request from the quality assurance apparatus 130, that is, the request for re-imaging or the request for additional imaging. The request reception unit 306 sends information regarding the received request to the examination control unit 302. The examination control unit 302 generates examination information corresponding to the request based on the information regarding the received request. For example, the examination control unit 302 extracts examination information stored in the storage unit 304 based on the image ID included in the received request for re-imaging and generates examination information including the same imaging procedure as that used for the imaging associated with the image ID. Alternatively, the examination control unit 302 extracts examination information stored in the storage unit 304 based on the examination ID included in the received request for additional imaging. Then, the examination control unit 302 generates examination information including the requested additional imaging based on the protocol ID included in the information regarding the received request.

After all the imaging operations included in the re-imaging or additional imaging have been finished, the imaging technician presses the examination end button 413. Then, the examination control unit 302 transmits the image data acquired by the re-imaging or additional imaging to the quality assurance apparatus 130 via the image transmission unit 305. Hereinafter, image data acquired based on a request for re-imaging is referred to as a re-acquired image. In addition, image data acquired on the basis of a request for additional imaging is referred to as an additionally acquired image.

The examination control unit 302 attaches the image ID included in the request for re-imaging to the re-acquired image. The examination control unit 302 issues a new image ID for the additionally acquired image and attaches the new image ID to the additionally acquired image. The new image ID is an image ID that enables image data to be distinguished from any other pieces of image data including pieces of image data that have been acquired based on examination requests received from the RIS 112 and pieces of image data acquired based on requests for re-imaging and additional imaging.

The examination control unit 302 also determines whether an examination completion notification has been transmitted for the examination for which re-imaging or additional imaging has been performed. If no examination completion notification has been made, the examination control unit 302 attaches the examination ID included in the information regarding the request to the image data. Then, the examination control unit 302 creates examination end information and causes the RIS transmission/reception unit 301 to transmit an examination completion notification including the examination end information to the RIS 112.

If the examination completion notification has been made, the examination control unit 302 issues a new examination ID and attaches the new examination ID to the image data. Then, the examination control unit 302 creates examination end information based on the new examination ID and causes the RIS transmission/reception unit 301 to transmit an examination completion notification including the examination end information to the RIS 112. The examination end information based on the new examination ID or the supplementary information including the new examination ID can include, as information regarding the related examination, the examination ID included in the information regarding the request for re-imaging or additional imaging.

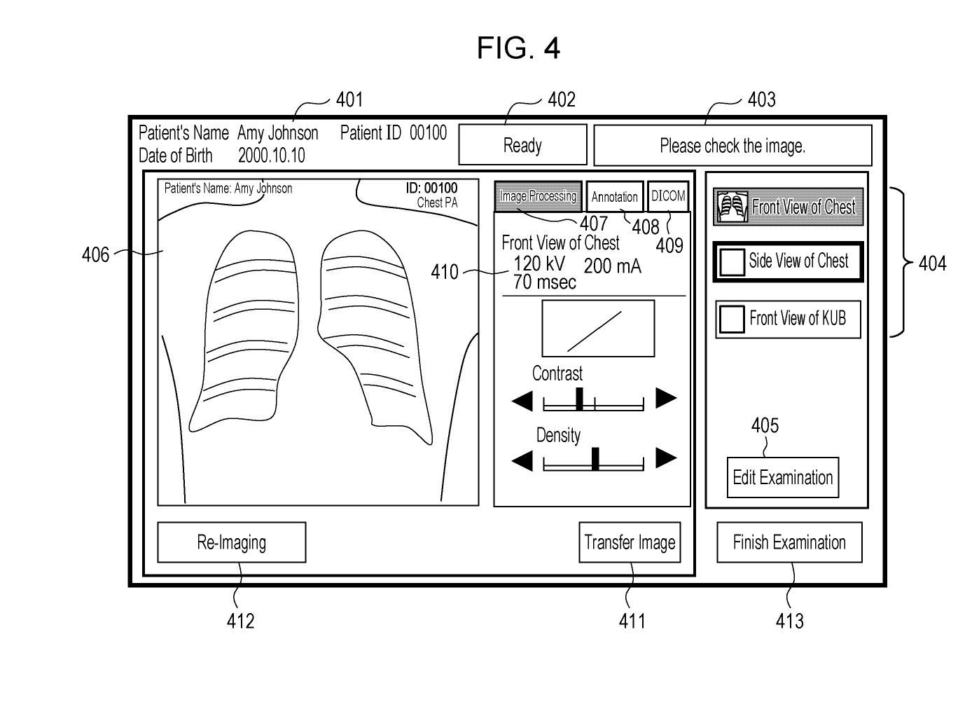

FIG. 4 illustrates an example of a GUI displayed on the display unit 11 of the imaging control apparatus 10 when an examination is performed. An input operation is performed on the GUI by using the input unit 12.

A patient information region 401 is a region in which patient information such as the patient's name, the patient ID, and the date of birth of the patient is displayed. A status region 402 is a region in which the statuses of the sensor unit 13 and the X-ray generation unit 14 are displayed. If both the sensor unit 13 and the X-ray generation unit 14 have finished preparations for imaging, information indicating that they are ready for imaging is displayed in the status region 402. If at least one of the sensor unit 13 and the X-ray generation unit 14 has not finished preparations for imaging, information indicating that they are not ready for imaging is displayed in the status region 402. A message display region 403 is a region in which a message to the imaging technician is displayed. For example, a message prompting the imaging technician to perform the next operation is displayed.

An imaging protocol region 404 is a region in which imaging protocols used in imaging scheduled to be performed are displayed in the requested order. In the example illustrated in FIG. 4, imaging protocols are displayed in the order of the chest front view, the chest side view, and the KUB (kidney, ureter, and bladder) front view. An examination edit button 405 is a button for editing examination information. In response to an input operation performed on the examination edit button 405, for example, a screen is displayed in which information such as the name of the imaging technician or the requesting department included in the examination information is edited or the order of the imaging protocols is modified.

An image region 406 is a region in which an acquired image is displayed. The imaging technician views the image displayed in the image region 406 after the imaging and determines whether the imaging has been appropriately performed.

An image processing tab 407 is a tab used to display a screen for performing image processing in an operation pane 410. Examples of image processing include adjustment of the density and the contrast of image data, enhancement processing, and geometric changes such as enlargement, reduction, and rotation. An annotation tab 408 is a tab used to display a screen for changing the position, the font size, and the items of the annotation in the operation pane 410. The annotation is desirably displayed so as to be superimposed on image data in accordance with the Grayscale Softcopy Presentation State (GSPS) of the DICOM standard. A DICOM tab 409 is a tab used to display a screen for displaying and correcting the values of DICOM tags in the operation pane 410. The imaging technician makes a correction appropriately via the screens displayed using these tabs.

An image transfer button 411 is a button used to transfer the acquired image data to the quality assurance apparatus 130 or the PACS 150 before the examination end button 413 is pressed. A re-imaging button 412 is a button used to perform re-imaging. For example, when the image is blurred because the subject has moved during imaging or the quality of the image is poor due to insufficient X-ray dose, re-imaging is performed. The examination end button 413 is a button used to end the examination. In response to an input operation performed on the examination end button 413, the image data acquired during the examination is transferred to the quality assurance apparatus 130 or the PACS 150.

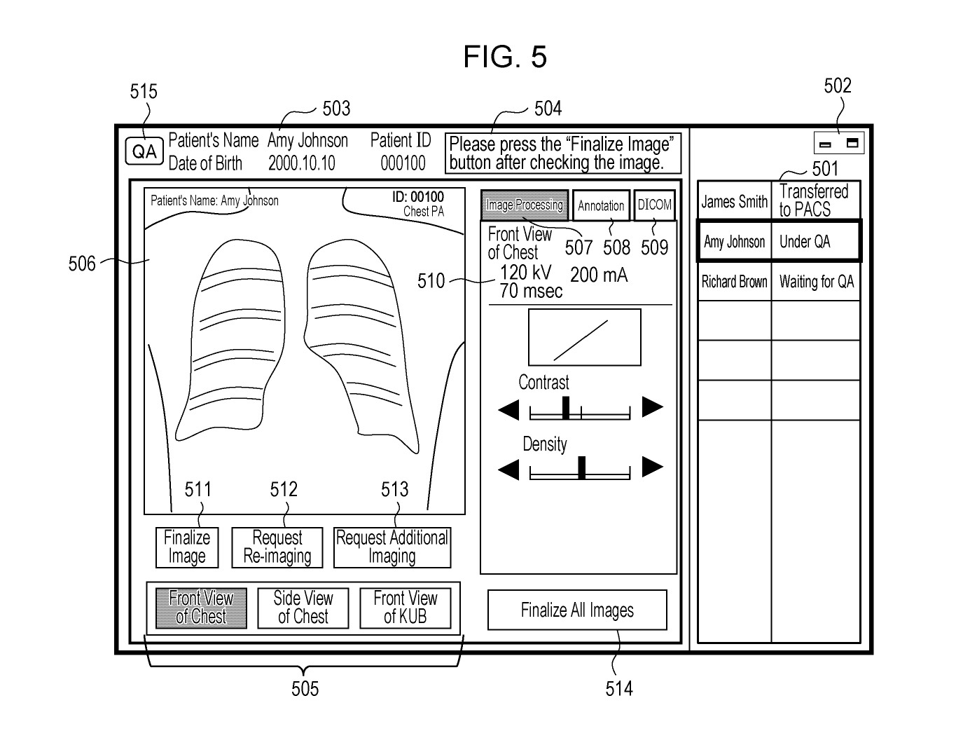

FIG. 5 is a diagram illustrating an example of a GUI displayed on a monitor (not illustrated) of the quality assurance apparatus 130.

A quality assurance target list 501 is a list of examinations that is received from external modalities, such as the modality 100, and for which the quality assurance operation is to be performed by using the quality assurance apparatus 130. The quality assurance target list 501 shows the patient's name, examination information, and the status of processing. In the example illustrated in FIG. 5, “Waiting for QA” is displayed as the processing status of an examination for which images on which the quality assurance operation is to be performed have been received from the modality 100 but the quality assurance operation has not been started. “Under QA” is displayed as the processing status of an examination for which an image based on the corresponding image data is displayed in an image region 506 and is currently subjected to the quality assurance operation.

If there is no examination whose processing status is “Under QA”, the processing status of the first examination assigned “Waiting for QA” can be automatically changed to “Under QA”. “Transferred to PACS” is displayed as the processing status of an examination for which the quality assurance operation has been completed and the image data has been transferred to the PACS 150. The quality assurance technician can perform the quality assurance operation by selecting a given examination from the quality assurance target list 501. Examinations for which the quality assurance operation has been completed among examinations displayed in the quality assurance target list 501 can be hidden or re-displayed by using a minimize button 502.

A patient information region 503 is a region in which patient information is displayed. Patient information includes, for example, the patient's name, the patient ID, and the date of birth of the patient. A message region 504 is a region in which a message to the quality assurance technician is displayed. For example, a message prompting the quality assurance technician to perform the next operation is displayed in the message region 504.

A performed protocol region 505 is a region in which protocols previously performed are displayed in requested order. The previously performed protocol is a protocol of each imaging operation that is included in an examination for which the quality assurance operation is performed and for which imaging has been completed by the modality. In the example illustrated in FIG. 5, protocols have been requested in the order of the chest front view, the chest side view, and the KUB front view, and the protocol of the chest front view is selected. The image region 506 is a region in which an image based on image data acquired in the selected previously performed protocol is displayed. The quality assurance technician observes the image displayed in the image region 506 and determines whether the image quality has reached a sufficiently high level to be used for medical diagnosis and treatment by doctors.

An image processing tab 507 is a tab used to display a screen for performing image processing in an operation pane 510. Examples of image processing include adjustment of the density and the contrast, enhancement processing, and geometric changes such as enlargement, reduction, and rotation. An annotation tab 508 is a tab used to display a screen for changing the position, the font size, and the items of the annotation in the operation pane 510. A DICOM tab 509 is a tab used to display a screen for displaying and correcting the values of DICOM tags in the operation pane 510. The quality assurance technician makes a correction appropriately via the screens displayed using these tabs.

The image finalize button 511 is a button used to complete the quality assurance operation and finalize the image data as image data to be transferred to the PACS 150. When the image finalize button 511 is pressed, the protocol for the chest side view is automatically selected from among the previously performed protocols displayed in the performed protocol region 505 and an image based on image data acquired in accordance with the protocol for the chest side view is displayed in the image region 506. The re-imaging request button 512 is a button used to request the modality to perform re-imaging. The additional imaging request button 513 is a button used to request the modality to perform additional imaging.

An all image finalize button 514 is a button used to complete the quality assurance operation of all the pieces of image data associated with the examination and finalize all the pieces of image data as pieces of image data to be transferred to the PACS 150. In addition, a quality assurance icon 515 indicates that the displayed image is an image on which the quality assurance operation needs to be performed. From the view point of the reliability of the operation, the all image finalize button 514 can be disabled unless the quality assurance operation is finished for all the images and all the pieces of image data to be transferred to the PACS 150 are finalized for an examination for which the quality assurance icon 515 is displayed. Alternatively, from the view point of the operation efficiency, all the pieces of image data can be finalized as pieces of image data to be transferred to the PACS 150 when the all image finalize button 514 is pressed.

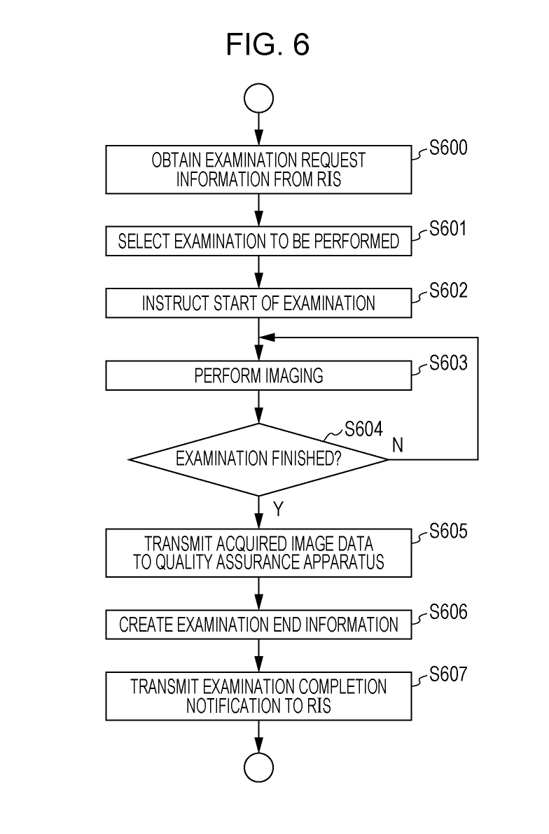

FIG. 6 is a flowchart illustrating an example of a series of processing steps performed by the imaging control apparatus 10 from reception of examination request information to termination of the examination. A detailed description is provided below.

In step S600, the RIS transmission/reception unit 301 requests the RIS 112 to transmit examination request information. This request is typically made at a timing when the imaging technician presses an examination acquisition button (not illustrated), which is a GUI displayed on the display unit 11 of the modality 100. In another example, the RIS transmission/reception unit 301 can automatically transmit the request at a certain interval or can transmit the request at a timing predetermined by the imaging technician. In response to the request transmitted to the RIS 112, the RIS transmission/reception unit 301 receives the examination request information transmitted from the RIS 112.

When a request to transmit examination request information is made, acquisition conditions are set for respective items included in the examination request information. The RIS transmission/reception unit 301 transmits at least one acquisition condition to the RIS 112 to request the RIS 112 to transmit examination request information. The RIS 112 that has requested by the modality 100 to transmit the examination request information extracts pieces of examination request information for which corresponding examinations have not been started and that meet the acquisition conditions from among pieces of examination request information stored therein and transmits the extracted pieces of examination request information to the modality 100. If no acquisition condition is set, the RIS 112 transmits all the pieces of examination request information for which corresponding examinations have not been started to the modality 100.

In step S601, the RIS transmission/reception unit 301 sends the acquired pieces of examination request information to the examination control unit 302. The examination control unit 302 creates a list of the received pieces of examination request information, causes the display unit 11 to display the list, and stores the pieces of examination request information in the storage unit 304. The imaging technician selects, by using the input unit 12, an examination to be started from the list of the pieces of examination request information displayed on the display unit 11. In response to an input operation performed by the imaging technician, the examination control unit 302 selects a piece of examination request information for which the examination is to be carried out.

In step S602, the examination control unit 302 controls the imaging unit 303 to start the examination. Specifically, in response to the imaging technician pressing an examination start button (not illustrated) for the examination selected in step S601, the examination control unit 302 causes the imaging unit 303 to start the examination.

In step S603, the imaging unit 303 performs imaging under the control of the examination control unit 302. The imaging unit 303 performs imaging in accordance with imaging protocols included in the examination being performed. At that time, the examination control unit 302 controls the RIS transmission/reception unit 301 to transmit an examination start notification to the RIS 112. In this way, the RIS transmission/reception unit 301 transmits an examination start notification to the RIS 112.

The imaging unit 303 controls the sensor unit 13 and the X-ray generation unit 14 and acquires information indicating whether the sensor unit 13 and the X-ray generation unit 14 are ready for imaging. If both the sensor unit 13 and the X-ray generation unit 14 have completed preparations for imaging, the acquired status is reflected in the status region 402, which is a GUI displayed on the display unit 11. The imaging technician confirms that the preparations for imaging have been completed through the content displayed in the status region 402 and presses the irradiation switch of the X-ray generation unit 14. In this way, the subject is irradiated with X-rays, and X-ray image data of the subject is acquired.

The examination control unit 302 performs correction processing and image processing on the acquired image data and stores the resultant image data in the storage unit 304. The examination control unit 302 attaches the image ID to the image data. The imaging technician checks an image displayed in the image region 406 based on the image data and determines whether the image is blurred because the patient has moved and whether imaging conditions have been appropriate. The imaging unit 303 performs imaging for all the imaging protocols requested in the examination request information.

In step S604, the examination control unit 302 determines whether the examination has finished. Specifically, the examination control unit 302 determines that the examination has finished if the imaging technician presses the examination end button 413 (Y in S604). The process then proceeds to step S605. If the imaging technician presses the re-imaging button 412 (N in S604), the examination control unit 302 generates examination information for re-imaging. The process then returns to step S603.

In step S605, the examination control unit 302 transmits the image data to an external apparatus via the image transmission unit 305. The external apparatus is, for example, the quality assurance apparatus 130 or the PACS 150. In the first embodiment, an image whose quality has been assured by using the quality assurance apparatus 130 is transmitted from the quality assurance apparatus 130 to the PACS 150.

In step S606, the examination control unit 302 creates examination end information based on the examination request information stored in the storage unit 304 and information regarding the imaging procedure performed by the imaging unit 303. The examination end information includes patient information and executed examination information, such as the examination ID, the date and time of the examination, the modality used in the examination, the name of the imaging technician who has carried out the examination, and the imaging conditions. In step S607, the RIS transmission/reception unit 301 transmits an examination completion notification including the examination end information to the RIS 112.

In the example illustrated in FIG. 6, steps S606 and S607 are described as processing that is performed if it is determined in step S604 that the examination has finished. However, the present embodiment is not limited to this configuration. A unit for making a notification regarding the end of an examination can be provided independently from an operation performed by the imaging technician to end the examination.

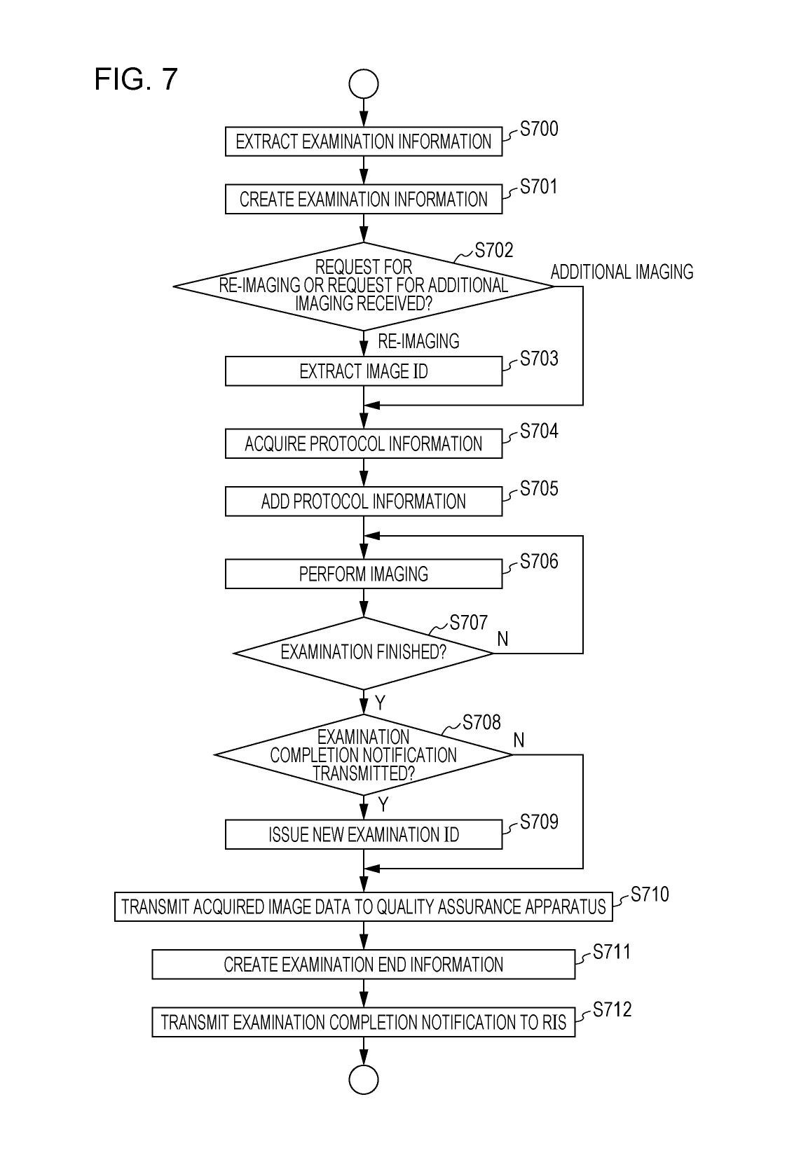

FIG. 7 is a flowchart illustrating an example of a process performed by the imaging control apparatus 10 that has received a request for re-imaging or additional imaging from an external apparatus. In step S605 illustrated in FIG. 6, the imaging control apparatus 10 transmits image data to the quality assurance apparatus 130. However, the quality assurance technician sometimes determines that the image data is not suitable to be used for medical diagnosis and treatment and requests the modality to perform re-imaging or additional imaging via a GUI illustrated in FIG. 5. The imaging control apparatus 10 performs an examination in response to a request for re-imaging or additional imaging from such an external apparatus. This process will be described in detail below.

In step S700, the examination control unit 302 extracts a piece of examination information relating to re-imaging or additional imaging requested by the quality assurance apparatus 130 from among pieces of examination information stored in the storage unit 304. Specifically, the request reception unit 306 receives a request for re-imaging or additional imaging from an external apparatus, such as the quality assurance apparatus 130. The request reception unit 306 sends information regarding the received request for re-imaging or additional imaging to the examination control unit 302. The examination control unit 302 extracts the corresponding examination information from the storage unit 304 by using the examination ID included in the information regarding the received request.

In step S701, the examination control unit 302 generates examination information for re-imaging or additional imaging based on the examination information extracted in step S700. The generated examination information does not include information regarding an imaging protocol for re-imaging or additional imaging.

In step S702, the examination control unit 302 determines whether the imaging request received from the quality assurance apparatus 130 is a request for re-imaging or a request for additional imaging. If the received request is a request for re-imaging, the process proceeds to step S703. If the received request is a request for additional imaging, the process proceeds to step S704.

In the first embodiment, the information regarding the request received from the quality assurance apparatus 130 includes information indicating whether the request is a request for re-imaging or a request for additional imaging. In the example illustrated in FIG. 5, the quality assurance technician presses the re-imaging request button 512 when making a request for re-imaging and presses the additional imaging request button 513 when making a request for additional imaging.

The imaging requesting unit 134 of the quality assurance apparatus 130 sets, for example, a re-imaging flag ON if the re-imaging request button 512 is pressed and then transmits information regarding the request to the imaging control apparatus 10. In addition, the imaging requesting unit 134 sets, for example, an additional imaging flag ON if the additional imaging request button 513 is pressed and transmits information regrading the request to the imaging control apparatus 10. The examination control unit 302 of the imaging control apparatus 10 determines the request is a request for re-imaging if the re-imaging flag is ON and determines the request is a request for additional imaging if an additional imaging flag is ON.

In step S703, the examination control unit 302 acquires the image ID of the image data for which re-imaging is to be performed from the information regarding the request for re-imaging received from the quality assurance apparatus 130. In step S704, the examination control unit 302 acquires protocol information indicating the imaging procedure to be performed for re-imaging or additional imaging. If it is determined in step S702 that the request is a request for re-imaging, the examination control unit 302 acquires, from the storage unit 304, the protocol ID of the imaging procedure that has been used for acquisition of the image data for which re-imaging is to be performed, based on the image ID acquired in step S703. If it is determined in step S702 that the request is a request for additional imaging, the examination control unit 302 acquires the protocol ID included in the information regarding the request.

In step S705, based on the protocol information acquired in step S704, protocol information indicating the imaging procedure to be performed for scheduled imaging is added to the examination information generated in step S701. In this way, examination information for re-imaging or additional imaging is generated by the examination control unit 302, so that re-imaging or additional imaging can be performed.

In step S706, the imaging unit 303 performs imaging in accordance with the examination information generated in step S705. Since processing to be performed by the imaging unit 303 for imaging is substantially the same as the processing of step S603 illustrated in FIG. 6, a detailed description thereof is omitted. The examination control unit 302 attaches supplementary information to the acquired image data.

If it is determined in step S702 that the request is a request for re-imaging, the examination control unit 302 attaches the image ID of the image for which re-imaging has been performed included in the information regrading the request from the quality assurance apparatus 130, as the image ID of the re-acquired image acquired in step S706. If it is determined in step S702 that the request is a request for additional imaging, the examination control unit 302 issues a new image ID and attaches the new image ID to the additionally acquired image acquired in step S706. With this configuration, when an external apparatus handles the image data acquired in step S706, the external apparatus can determine whether the image data is data of a re-acquired image or data of an additionally acquired image.

In step S707, the examination control unit 302 determines whether the examination of re-imaging or additional imaging has finished. Since the determination processing performed in step S707 is substantially the same as the processing of step S604 illustrated in FIG. 6, a detailed description thereof is omitted.

In step S708, the examination control unit 302 determines whether an examination completion notification has previously been transmitted from the imaging control apparatus 10 to the RIS 112 for the examination for which re-imaging or additional imaging has been requested. If the examination completion notification has previously been transmitted to the RIS 112 (Y in S708), the process proceeds to step S709. If the examination completion notification has not been transmitted to the RIS 112 yet (N in S708), the process proceeds to step S710.

In step S709, the examination control unit 302 issues a new examination ID as an ID for uniquely identifying the examination including the re-imaging or additional imaging. In this way, the inconsistency of information, which is caused as a result of transmission of new image data for an examination for which the examination completion notification has previously been transmitted to the RIS 112, is successfully avoided.

In step S710, the image transmission unit 305 transmits, to an external apparatus, the image data acquired in step S706. In the first embodiment, the image transmission unit 305 transmits the image data to the quality assurance apparatus 130 in step S710. Since the processing of step S710 is substantially the same as the processing of step S605 illustrated in FIG. 6, a detailed description thereof is omitted.

In step S711, the examination control unit 302 re-creates examination end information. If a new examination ID has been issued in step S709, the examination end information includes only information regarding the imaging requested by the quality assurance apparatus 130. If it is determined in step S708 that the examination completion notification has not been transmitted to the RIS 112, the examination ID of the examination for which re-imaging or additional imaging has been performed is used. The examination end information created by the examination control unit 302 includes information regarding the previous imaging performed before the request has been made and the requested imaging.

In step S712, the RIS transmission/reception unit 301 transmits an examination completion notification to the RIS 112. Since the processing of step S712 is substantially the same as the processing of step S607 illustrated in FIG. 6, a detailed description thereof is omitted.

As described above, the modality 100 can appropriately generate examination information in response to a request for re-imaging or additional imaging made by the quality assurance apparatus 130. In addition, the same image ID as that of image data for which re-imaging has been performed is attached to a re-acquired image. In this way, the acquired image data can be transmitted as image data that replaces the image data for which the re-imaging has been requested. If an examination completion notification has been made for an examination for which re-imaging or additional imaging has been requested, a new examination ID is issued. In this way, the inconsistency of information caused by re-imaging or additional imaging is successfully avoided in the intra-hospital information system, such as the HIS 111 or the RIS 112.

### Second Embodiment

An information processing apparatus according to a second embodiment generates examination information for re-imaging or additional imaging based on the examination information received from an external apparatus.

In the first embodiment, a description was provided of the example case where examination information and image data of an examination for which re-imaging or additional imaging is requested are stored in the storage unit 304 of the modality 100. However, there can be cases where the modality 100 receives a request for re-imaging or additional imaging and performs re-imaging or additional imaging for an examination that has been performed by the modality 101. In the second embodiment, a description will be provided of an example case where information such as examination information and image data is acquired from an external apparatus, which is different from a management apparatus such as the RIS 112, based on a request for re-imaging or additional imaging received from the external apparatus and then examination information for the re-imaging or additional imaging is generated.

The information control apparatus according to the second embodiment is, for example, the imaging control apparatus 10 of the modality 100. Since the configuration and the GUIs of the imaging control apparatus 10 are substantially the same as those illustrated in FIGS. 2, 3, 4, and 6, a detailed description thereof is omitted. In addition, since the configuration and the GUIs of the quality assurance apparatus 130 are substantially the same as those illustrated in FIGS. 3 and 5, a detailed description thereof is omitted.

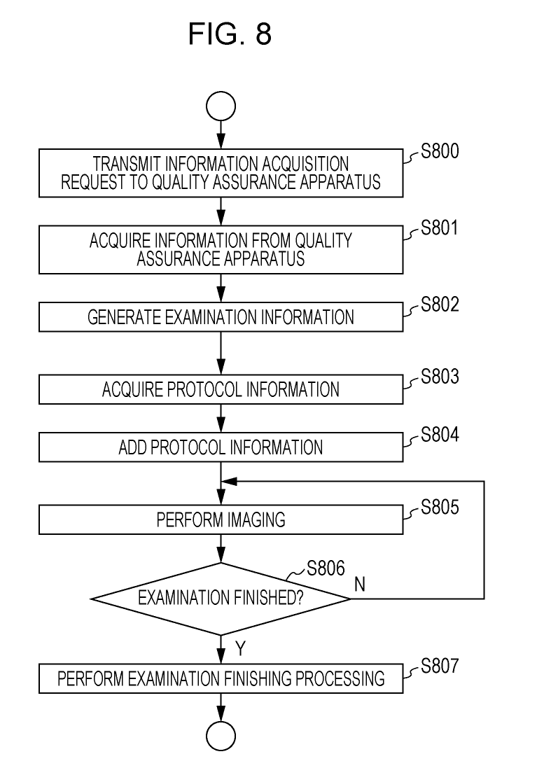

FIG. 8 is a flowchart illustrating an example of a process performed by the imaging control apparatus 10 from reception of a request for re-imaging or additional imaging to termination of the examination.

The request reception unit 306 of the modality 100 receives a request for re-imaging or additional imaging from the quality assurance apparatus 130. The request reception unit 306 sends information re-grading the request to the examination control unit 302.

In step S800, the supplementary information acquisition unit 307 requests the external apparatus to transmit examination information of an examination for which the request for re-imaging or additional imaging has been made. In the second embodiment, the external apparatus is the quality assurance apparatus 130. For example, the supplementary information acquisition unit 307 requests the quality assurance apparatus 130 to transmit supplementary information, such as the examination ID, the image ID, and the protocol ID included in the information regarding the request.

In step S801, the supplementary information acquisition unit 307 acquires supplementary information requested in step S800. Specifically, the supplementary information management unit 131 of the quality assurance apparatus 130 receives the request transmitted in step S800. The supplementary information management unit 131 identifies an examination and an image for which re-imaging or additional imaging is to be performed, based on the request received from the imaging control apparatus 10 and transmits supplementary information corresponding to the request to the imaging control apparatus 10. The supplementary information acquisition unit 307 sends, to the examination control unit 302, information such as the examination ID, the image ID, and the protocol ID received from the quality assurance apparatus 130.

In step S802, the examination control unit 302 generates examination information for re-imaging or additional imaging. The processing of step S802 is substantially the same as that of step S701 illustrated in FIG. 7.

In step S803, the examination control unit 302 acquires protocol information indicating the imaging procedure to be performed for re-imaging or additional imaging. The examination control unit 302 acquires the protocol ID representing the imaging procedure for imaging to be performed, based on the information acquired in step S801 as in the processing of step S704 illustrated in FIG. 7.

In step S804, the examination control unit 302 adds the protocol information representing the imaging procedure to be used for the scheduled imaging to the examination information generated in step S802, based on the protocol information acquired in step S803. In step S805, the imaging unit 303 performs imaging based on the examination information generated in step S804. Since the processing of this step is substantially the same as the processing of step S706 illustrated in FIG. 7, a detailed description thereof is omitted.

In step S806, the examination control unit 302 determines whether the examination including the re-imaging or additional imaging has finished. Since the determination processing of step S806 is substantially the same as that of step S604 illustrated in FIG. 6, a detailed description thereof is omitted. If it is determined that the examination has finished (Y in S806), the process proceeds to step S807. If it is determined that the examination has not finished (N in S806), the process returns to step S805.

In step S807, the examination control unit 302 performs processing for ending the examination including the re-imaging or additional imaging. Since the processing of step S807 is substantially the same as the processing from step S708 to step S712 illustrated in FIG. 7, a detailed description thereof is omitted.

As described above, the imaging control apparatus 10 can acquire the image ID, the examination ID, and the protocol ID, which are supplementary information used to perform re-imaging or additional imaging, from an external apparatus and generating examination information for re-imaging or additional imaging.

An example where the imaging control apparatus 10 requests an external apparatus to transmit supplementary information upon receipt of a request for re-imaging or additional imaging from the external apparatus has been described above. However, the present embodiment is not limited to this example. For example, the examination control unit 302 can determine whether examination information corresponding to the request from the external apparatus is stored in the storage unit 304. If the corresponding examination information is stored in the storage unit 304, the examination control unit 302 can perform the process described in the first embodiment. If the corresponding examination information is not stored in the storage unit 304, the examination control unit 302 can perform the process illustrated in FIG. 8.

### Third Embodiment

An information processing apparatus according to a third embodiment receives a request for re-imaging or additional imaging, acquires examination information from the RIS 112, and generates examination information for the re-imaging or additional imaging.

The information processing apparatus according to the third embodiment is the imaging control apparatus 10 of the modality 100. Since the configuration and GUIs of the imaging control apparatus 10 are substantially the same as those illustrated in FIGS. 2, 3, 4, and 6, a detailed description thereof is omitted.

The RIS transmission/reception unit 301 communicates with the RIS 112 and receives examination request information. Specifically, when requesting the RIS 112 to provide the examination request information, the RIS transmission/reception unit 301 transmits, to the RIS 112, patient information such as the patient ID and the patient's name, examination information such as the examination ID, and imaging information such as the protocol ID as search conditions. According to the Modality Worklist Management of DICOM, information included in the examination information acquisition request is handled as examination extraction conditions in the RIS 112.

The RIS 112 that has received the request including the search conditions from the RIS transmission/reception unit 301 generates examination request information that satisfies the conditions and transmits the examination request information to the imaging control apparatus 10. Since the configuration and the GUIs of the RIS transmission/reception unit 301 are substantially the same as those illustrated in FIGS. 2, 3, 4, and 6, a detailed description thereof is omitted. The RIS transmission/reception unit 301 receives the examination request information transmitted from the RIS 112. The RIS transmission/reception unit 301 sends the received examination request information to the examination control unit 302 and the storage unit 304.

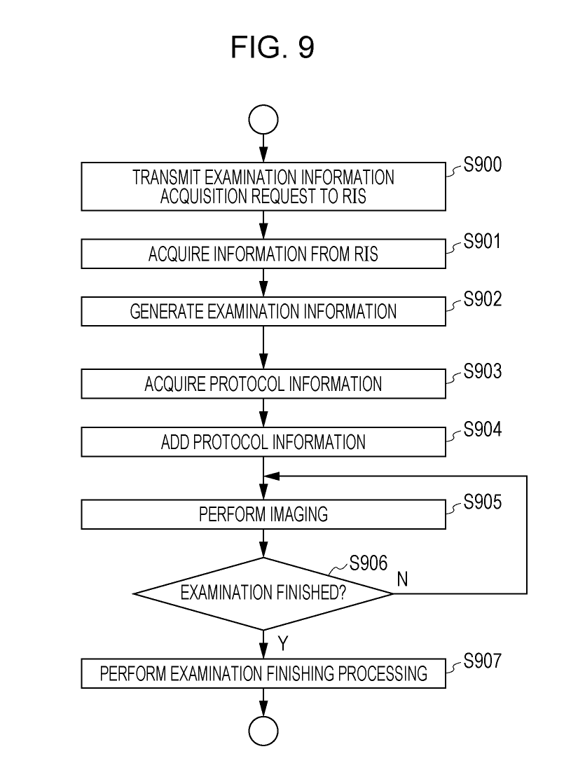

FIG. 9 is a flowchart illustrating an example of a process performed by the imaging control apparatus 10 that has received a request for re-imaging or additional imaging from an external apparatus until it finishes the examination including the re-imaging or additional imaging.

The request reception unit 306 of the modality 100 acquires a request for re-imaging or additional imaging from the quality assurance apparatus 130. The request reception unit 306 sends information regarding the request to the examination control unit 302.

In step S900, the examination control unit 302 transmits, via the RIS transmission/reception unit 301 to the RIS 112, an examination information acquisition request including search conditions such as information regarding the patient ID, the examination ID, and the protocol ID that are included in the request for re-imaging or additional imaging.

In step S901, the RIS transmission/reception unit 301 acquires the information from the RIS 112. Specifically, the RIS 112 transmits, to the modality 100, examination information corresponding to the examination information acquisition request transmitted from the modality 100 in step S900. The RIS transmission/reception unit 301 receives the examination information transmitted from the RIS 112 and sends the examination information to the examination control unit 302.

In step S902, the examination control unit 302 generates examination information for re-imaging or additional imaging. The processing of step S902 is substantially the same as that of step S701 illustrated in FIG. 7.

In step S903, the examination control unit 302 acquires protocol information indicating the imaging procedure to be performed for the re-imaging or additional imaging. The examination control unit 302 acquires the protocol ID indicating the imaging procedure for the imaging to be performed, based on the information acquired in step S901 as in the processing of step S704 illustrated in FIG. 7.

In step S904, the examination control unit 302 adds the protocol information indicating the imaging procedure to be used in the scheduled imaging to the examination information generated in step S902, based on the protocol information acquired in step S903. In this way, examination information for re-imaging or additional imaging is generated by the examination control unit 302, so that re-imaging and additional imaging can be performed. The imaging technician can modify the information acquired from the RIS 112 if necessary and add the protocol information for re-imaging or additional imaging.

In step S905, the imaging unit 303 performs imaging based on the examination information generated in step S904. Since this step is substantially the same as the processing of step S706 illustrated in FIG. 7, a detailed description thereof is omitted.

In step S906, the examination control unit 302 determines whether the examination including the re-imaging or additional imaging has finished. Since the determination processing of step S906 is substantially the same as the processing of step S604 illustrated in FIG. 6, a detailed description thereof is omitted. If it is determined that the examination has finished (Y in S906), the process proceeds to step S907. If it is determined that the examination has not finished (N in S906), the process returns to step S905.

In step S907, the examination control unit 302 performs processing for ending the examination including the re-imaging or additional imaging. Since the processing of step S907 is substantially the same as the processing from step S708 to step S712 illustrated in FIG. 7, a detailed description thereof is omitted.

As described above, the imaging control apparatus 10 can acquire, from the RIS 112, the image ID, the examination ID, and the protocol ID that are supplementary information used to perform re-imaging or additional imaging and generating examination information for the re-imaging or additional imaging. In particular, since a mechanism for requesting the RIS 112 to provide examination information is already utilized, the request can easily be made.

### Modifications

In the first to third embodiments, a description has been provided of an example where it is determined whether information of the request indicates re-imaging or additional imaging based on information, such as the re-imaging flag or the additional imaging flag, included in the information of the imaging request received from an external apparatus. However, this is not seen to be limiting.

It can be determined whether the request is a request for re-imaging or a request for additional imaging based on other information included in the information of the imaging request received from the external apparatus. For example, it is determined that the request is a request for re-imaging if the imaging request received from the external apparatus includes the examination ID and the image ID. Since re-imaging is intended to re-acquire a specific image, re-imaging can be performed if an image to be re-taken is identified. In addition, it is determined that the request is a request for additional imaging if the imaging request received from the external apparatus includes the examination ID and the protocol ID. Since additional imaging is intended to additionally acquire a new image for a specific examination, additional imaging requires information regarding an examination to which a protocol ID is added and information regarding the imaging procedure to be added.

A program that implements one or more functions of the above-described embodiments is supplied to a system or apparatus via a network or storage medium, and one or more processors in a computer of the system or apparatus reads and executes the program to perform a process. The embodiments can be implemented through such a process. In addition, the embodiments can be implemented by a circuit (for example, an application specific integrated circuit (ASIC)) that implements one or more functions.

The information processing apparatus according to each of the embodiments described above can be implemented as a single apparatus, or a plurality of apparatuses that communicate with one another can perform the processes described above. Both of these configurations are included in the embodiments. The processes described above can be performed by a common server or servers. The information processing apparatus and the plurality of apparatuses constituting the information processing system are only required to be able to communicate with one another at a predetermined communication rate and need not necessarily exist in the same facility or the same country.

Embodiments include an embodiment in which a software program that implements the functions of the embodiments described above is supplied to a system or apparatus and a computer of the system or apparatus reads and executes code of the supplied program.

Accordingly, program code that is installed to the computer so as to implement the processes according to the embodiments by using the computer is one embodiment. In addition, an operating system (OS) or the like that is running in the computer performs some or all of the processes based on instructions included in the program read by the computer and the functions of the embodiments described above can be implemented through such processes.

Any given combinations of the embodiments described above are applicable.

According to an embodiment, appropriate information is successfully attached to a medical image acquired based on a request for re-imaging or additional imaging made from an external apparatus. Consequently, the workflow can be improved for the user who utilizes medical images acquired by re-imaging or additional imaging.

### Other Embodiments

Embodiment(s) can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While exemplary embodiments have been described, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-091610 filed Apr. 28, 2016, which is hereby incorporated by reference herein in its entirety.

What is claimed is:

1. An information processing apparatus comprising:
a first acquiring unit configured to acquire a request for re-imaging or a request for additional imaging, the request for re-imaging including information for requesting imaging for acquiring a second medical image as re-imaging for a first medical image previously acquired, the request for additional imaging including information for requesting imaging for additionally acquiring a third medical image; and
an attaching unit configured to:
attach, to the second medical image, information for identifying the first medical image for which the request for re-imaging has been made as information for identifying the second medical image in a case where the request for re-imaging is acquired by the first acquiring unit, and
attach, to the third medical image, information for identifying the third medical image in a case where the request for additional imaging is acquired by the first acquiring unit.

2. The information processing apparatus according to claim 1, further comprising:
a second acquiring unit configured to acquire information indicating whether a completion notification has been output to a management apparatus in relation to an examination for which the request for re-imaging or the request for additional imaging has been acquired, wherein the management apparatus manages information regarding an examination including imaging for acquiring medical images, wherein the completion notification indicates that imaging for acquiring all the medical images included in the examination has completed, and
wherein the attaching unit:
attaches, to the second medical image or the third medical image, as information for identifying an examination including imaging for acquiring the second medical image or the third medical image, information for distinguishing the examination from the examination for which the completion notification has been output in a case where the completion notification has been output to the management apparatus in relation to the examination for which the request for re-imaging or the request for additional imaging has been acquired, and
attaches, to the second medical image or the third medical image, as information for identifying the examination including imaging for acquiring the second medical image or the third medical image, information for identifying the examination for which the request for re-imaging or the request for additional imaging has been acquired in a case where the completion notification has not been output to the management apparatus in relation to the examination for which the request for re-imaging or the request for additional imaging has been acquired.

3. The information processing apparatus according to claim 2, wherein the request for re-imaging includes at least information for identifying the examination including imaging for acquiring the first medical image and information for identifying the first medical image.

4. The information processing apparatus according to claim 1, wherein the request for additional imaging includes at least information for identifying an examination to which imaging for acquiring the third medical image is added and information for identifying an imaging procedure used for imaging for acquiring the third medical image.

5. The information processing apparatus according to claim 1, further comprising:
a generating unit configured to:
generate examination information used to perform imaging for acquiring the second medical image based on the request for re-imaging acquired by the first acquiring unit, and
generate examination information used to perform imaging for acquiring the third medical image based on the request for additional imaging acquired by the first acquiring unit.

6. The information processing apparatus according to claim 2, further comprising:
a generating unit configured to:
generate examination information used to perform imaging for acquiring the second medical image based on information that is acquired from the management apparatus based on the request for re-imaging acquired by the first acquiring unit, and
generate examination information used to perform imaging for acquiring the third medical image based on information that is acquired from the management apparatus based on the request for additional imaging acquired by the first acquiring unit.

7. The information processing apparatus according to claim 5, wherein the generating unit acquires information attached to a medical image acquired during an examination identified based on examination identification information included in the request for re-imaging or the request for additional imaging and generates the examination information.

8. The information processing apparatus according to claim 6, wherein the generating unit acquires, from an external apparatus different from the management apparatus, information attached to a medical image acquired during an examination identified based on examination identification information included in the request for re-imaging or the request for additional imaging and generates the examination information.

9. The information processing apparatus according to claim 1, further comprising:
a determining unit configured to determine whether the information acquired by the first acquiring unit is the request for re-imaging or the request for additional imaging.

10. The information processing apparatus according to claim 9, wherein the determining unit determines whether the information acquired by the first acquiring unit is the request for re-imaging or the request for additional imaging based on information included in the information acquired by the first acquiring unit.

11. The information processing apparatus according to claim 1, further comprising:
an image acquiring unit configured to acquire a medical image based on a request for imaging from a management apparatus and the request for re-imaging or the request for additional imaging acquired by the first acquiring unit, wherein the management apparatus manages information regarding an examination including imaging for acquiring medical images; and
an output unit configured to output the medical image acquired by the image acquiring unit to an external apparatus together with the information attached by the attaching unit.

12. An information processing system comprising:
a first acquiring unit configured to acquire a request for re-imaging or a request for additional imaging, the request for re-imaging including information for requesting imaging for acquiring a second medical image as re-imaging for a first medical image previously acquired, the request for additional imaging including information for requesting imaging for additionally acquiring a third medical image; and
an attaching unit configured to:
attach, to the second medical image, information for identifying the first medical image for which the request for re-imaging has been made as information for identifying the second medical image in a case where the request for re-imaging is acquired by the first acquiring unit, and
attach, to the third medical image, information for identifying the third medical image in a case where the request for additional imaging is acquired by the first acquiring unit.

13. An information processing method comprising:
a first acquiring step of acquiring a request for re-imaging or a request for additional imaging, the request for re-imaging including information for requesting imaging for acquiring a second medical image as re-imaging for a first medical image previously acquired, the request for additional imaging including information for requesting imaging for additionally acquiring a third medical image; and
an attaching step of
attaching, to the second medical image, information for identifying the first medical image for which the request for re-imaging has been made as information for identifying the second medical image in a case where the request for re-imaging is acquired in the first acquiring step, and
attaching, to the third medical image, information for identifying the third medical image in a case where the request for additional imaging is acquired in the first acquiring step.

14. A non-transitory computer readable storage medium storing computer executable instructions for causing a computer to execute:
a first acquiring process of acquiring a request for re-imaging or a request for additional imaging, the request for re-imaging including information for requesting imaging for acquiring a second medical image as re-imaging for a first medical image previously acquired, the request for additional imaging including information for requesting imaging for additionally acquiring a third medical image; and
an attaching process of
attaching, to the second medical image, information for identifying the first medical image for which the request for re-imaging has been made as information for identifying the second medical image in a case where the request for re-imaging is acquired in the first acquiring process, and
attaching, to the third medical image, information for identifying the third medical image in a case where the request for additional imaging is acquired in the first acquiring process.
